SEQUENCE LISTING (4 sequences)

SEQ ID NO: 1 - DNA, 1442 bp, Pseudomonas sp.
tgcaagtcgagcggtagagaggtgcttgcacctcttgagagcggcggacgggtgagtaatgcctaggaatctgcctggtagtgggggataacgctcggaaacggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttagtgttttgacgttaccgacagaataagcaccggctaactctgtgccagcagccgcggtaatacagagggtgcaagcgttaatcggaattactgggcgtaaagcgcgcgtaggtggtttgttaagttggatgtgaaagccccgggctcaacctgggaactgcattcaaaactgacaagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcggggggacggttaccacggtgtgattcatgactggggtgaagtcgtaaca

SEQ ID NO: 2 - DNA, 1442 bp, Pseudomonas sp.; identical to SEQ ID NO: 1

SEQ ID NO: 3 - DNA, 1439 bp, Pseudomonas sp.
tgttacgacttcaccccagtcatgaatcacaccgtggtaaccgtcccccgaaggttagactagctacttctggtgcaacccactcccatggtgtgacgggcggtgtgtacaaggcccgggaacgtattcaccgcgacattctgattcgcgattactagcgattccgacttcacgcagtcgagttgcagactgcgatccggactacgatcggttttatgggattagctccacctcgcggcttggcaaccctctgtaccgaccattgtagcacgtgtgtagcccaggccgtaagggccatgatgacttgacgtcatccccaccttcctccggtttgtcaccggcagtctccttagagtgcccaccataacgtgctggtaactaaggacaagggttgcgctcgttacgggacttaacccaacatctcacgacacgagctgacgacagccatgcagcacctgtctcaatgttcccgaaggcaccaatccatctctggaaagttcattggatcaaggcctggtaaggttcttcgcgttgcttcgaattaaaccacatgctccaccgcttgtgcgggcccccgtcaattcatttgagttttaaccttgcggccgtactccccaggcggtcaacttaatgcgttagctgcgccactaagagctcaaggctcccaacggctagttgacatcgtttacggcgtggactaccagggtatctaatcctgtttgctccccacgctttcgcacctcagtgtcagtatcagtccaggtggtcgccttcgccactggtgttccttcctatatctacgcatttcaccgctacacaggaaattccaccaccctctaccatactctagcttgtcagttttgaatgcagttcccaggttgagcccggggctttcacatccaacttaacaaaccacctacgcgcgctttacgcccagtaattccgattaacgcttgcaccctctgtattaccgcggctgctggcacagagttagccggtgcttattctgtcggtaacgtcaaaacactaacgtattaggttaatgcccttcctcccaacttaaagtgctttacaatccgaagaccttcttcacacacgcggcatggctggatcaggctttcgcccattgtccaatattccccactgctgcctcccgtaggagtctggaccgtgtctcagttccagtgtgactgatcatcctctcagaccagttacggatcgtcgccttggtgagccattacctcaccaactagctaatccgacctaggctcatctgatagcgcaaggcccgaaggtcccctgctttctcccgtaggacgtatgcggtattagcgtccgtttccgagcgttatcccccactaccaggcagattcctaggcattactcacccgtccgccgctctcaagaggtgcaagcacctctctaccgctcgacttgca

SEQ ID NO: 4 - DNA, 1446 bp, Pseudomonas sp.
tgttacgacttcaccccagtcatgaatcacaccgtggtaaccgtcccccgaaggttagactagctacttctggtgcaacccactcccatggtgtgacgggcggtgtgtacaaggcccgggaacgtattcaccgcgacattctgattcgcgattactagcgattccgacttcacgcagtcgagttgcagactgcgatccggactacgatcggttttatgggattagctccacctcgcggcttggcaaccctctgtaccgaccattgtagcacgtgtgtagcccaggccgtaagggccatgatgacttgacgtcatccccaccttcctccggtttgtcaccggcagtctccttagagtgcccaccataacgtgctggtaactaaggacaagggttgcgctcgttacgggacttaacccaacatctcacgacacgagctgacgacagccatgcagcacctgtctcaatgttcccgaaggcaccaatccatctctggaaagttcattggatgtcaaggcctggtaaggttcttcgcgttgcttcgaattaaaccacatgctccaccgcttgtgcgggcccccgtcccgtcaattcatttgagttttaaccttgcggccgtactccccaggcggtcaacttaatgcgttagctgcgccactaagagctcaaggctcccaacggctagttgacatcgtttacggcgtggactaccagggtatctaatcctgtttgctccccacgctttcgcacctcagtgtcagtatcagtccaggtggtcgccttcgccactggtgttccttcctatatctacgcatttcaccgctacacaggaaattccaccaccctctaccatactctagcttgtcagttttgaatgcagttcccaggttgagcccggggctttcacatccaacttaacaaaccacctacgcgcgctttacgcccagtaattccgattaacgcttgcaccctctgtattaccgcggctgctggcacagagttagccggtgcttattctgtcggtaacgtcaaaacactaacgtattaggttaatgcccttcctcccaacttaaagtgctttacaatccgaagaccttcttcacacacgcggcatggctggatcaggctttcgcccattgtccaatattccccactgctgcctcccgtaggagtctggaccgtgtctcagttccagtgtgactgatcatcctctcagaccagttacggatcgtcgccttggtgagccattacctcaccaactagctaatccgacctaggctcatctgatagcgcaaggcccgaaggtcccctgctttctcccgtaggacgtatgcggtattagcgtccgtttccgagcgttatcccccactaccaggcagattcctaggcattactcacccgtccgccgctctcaagaggtgcaagcacctctctaccgctcgacttgca

(19) United States
(12) Patent Application Publication
(10) Pub. No.: US 2024/0000032 A1
(43) Pub. Date: Jan. 4, 2024

(54) Bioprotectant Endophytes of Cannabis

(71) Applicant: Agriculture Victoria Services PTY LTD, Bundoora, VICTORIA (AU)

(72) Inventors: Piyumi Niroshini Ekanayake, Doreen (AU); Dilani De Silva, South Morang (AU); Jatinder Kaur, Taylors Hill (AU); Tongda Li, Southbank (AU); Ross Mann, Coburg (AU); Timothy Ivor Sawbridge, Coburg (AU); German Carlos Spangenberg, Bundoora (AU)

(73) Assignee: Agriculture Victoria Services PTY LTD, Bundoora, VICTORIA (AU)

(21) Appl. No.: 18/039,651

(22) PCT Filed: Dec. 9, 2021
(86) PCT No.: PCT/AU2021/051471

§ 371 Date: May 31, 2023

(30) Foreign Application Priority Data
Dec. 10, 2020 (AU) 2020904592

(51) Int. Cl. A01H 3/00; A01H 17/00; A01H 6/28; A01H 5/12; C12N 1/20; A01N 63/27; A01P 3/00

(52) U.S. Cl. CPC A01H 3/00; A01H 17/00; A01H 6/28; A01H 5/12; C12N 1/205; A01N 63/27; A01P 3/00; C12R 2001/38

(57) ABSTRACT

The present invention relates to novel endophytes of plants of the Cannabaceae family, particularly bioprotective Pseudomonas sp. endophytes, and also to plants and parts thereof infected therewith, and related methods, including methods for conferring bioprotection to plants and for selecting a bioprotectant endophyte of a plant of the Cannabaceae family.

### FIELD OF THE INVENTION

The present invention relates to novel bioprotectant endophytes of plants of the Cannabaceae family, plants and parts thereof infected therewith and related methods, including methods for conferring bioprotection to plants and for selecting a bioprotectant endophyte of a plant of the Cannabaceae family.

### BACKGROUND OF THE INVENTION

Efficient and consistent production of agricultural products in sufficient quantities, particularly those going to food and medicine, is a world-wide challenge. The field of terrestrial agriculture is relied upon to produce vast supplies of the world's food and medicinal products and textiles.

Management of the economics, logistics and sheer scale of agricultural output is a considerable undertaking. However, consumers continue to demand higher quality products against the constant challenges faced by farmers in the production itself. These challenges include for example the inherent susceptibility of crops to climatic conditions, and many other abiotic and biotic stresses, such as invertebrate pests and microbe and viral crop infections.

While there is no one solution to all of these issues, there are significant gains to be achieved from improvements in any one area, one of which in particular is the protection of crops from pathogenic microbial infection.

Microbial infection of agricultural crops for food, medicine and textiles may lead to blight, taint or spoilage and partial or total loss of harvest and end product. For instance, microbes and their associated metabolic activities can degrade harvestable products, generate unpleasant odours, flavours and appearances in food, and even be allergenic or toxic to humans and/or animals e.g. when ingested, often through the production of mycotoxins.

One specific example concerns the Cannabis plant. Presently, it is believed that there are three distinct species in the genus: Cannabis sativa, Cannabis indica and Cannabis ruderalis. Cannabis and its extracts and derivatives such as hashish is a widely used drug around the world and is increasingly being recognised in the treatment of a range of medical conditions such as epilepsy, multiple sclerosis and chronic pain. While Cannabis remains illicit in many countries, recent passed and proposed legislation seeks to legalise it for medical purposes, and in some countries even recreational use. Further, hemp forms of Cannabis are also used to produce fibrous materials. Consequently, with increasing demand requires increased production which places significant focus on the need for improved crop production and thus protection from pathogenic microbes. One pathogenic microbe of Cannabis is the fungus Botrytis cinerea, which can cause grey mould and result in a tainted or spoiled product.

Another example is in viticulture. Grape is an even older known cultivated crop and one of the world's most significant food products. The most common species is Vitis vinefera—the common grape vine—which is cultivated around the world, especially for winemaking. Grape production is susceptible to many factors including significantly pathogenic infection. The same fungus Botrytis cinerea is pathogenic to grape vines, particularly the fruit, and can cause in particular Botrytis bunch rot in viticulture. This can result in a spoiled food product, and often a harvest must be discarded. Climate plays a significant role in the prevalence and vigour of B. cinerea infection, though climate is one factor which can rarely be controlled. This fungus also affects various other crops, including for example strawberry and tomato.

Fusarium proliferatum is another pathogenic fungus which can detrimentally affect and spoil many of the world's most significant crops, including Cannabis and grapes, among others.

Fusarium species are also commonly associated with the production of mycotoxins in food products potentially leading to taint and spoilage.

While chemical fungicides are widely used to combat pathogenic fungal infections, this brings forth an associated set of drawbacks including health risks, environmental contamination and the increasing incidence of fungicide resistance. Further, in the case of medicinal Cannabis cultivation, no chemical fungicide can be used, leaving the crop exposed to infection by B. cinerea and F. proliferatum without an alternative effective solution.

Consequently, there exists a need to overcome, or at least alleviate, one or more of the difficulties or deficiencies associated with the prior art, and in particular there remains a need for improved crop protection and production and methods that assist in the ongoing search for preventative measures of pathogenic microbe proliferation.

### SUMMARY OF THE INVENTION

In one aspect, the present invention provides a substantially purified or isolated Pseudomonas sp. endophyte of a plant of the Cannabaceae family.

By “endophyte” is meant an organism, generally a micro-organism that co-exists in a mutually beneficial relationship with a plant. Endophytes generally live on, in, or otherwise in close proximity to a plant and rely on the plant for survival, while at the same time confer a certain benefit to the plant. Endophytes of Pseudomonas sp. are bacterial endophytes.

By “substantially purified” in the context of an endophyte is meant that the endophyte is free of other organisms. The term includes, for example, an endophyte in axenic culture.

Preferably, the endophyte is at least approximately 90% pure, more preferably at least approximately 95% pure, even more preferably at least approximately 98% pure, even more preferably at least approximately 99% pure.

By “isolated” in the context of an endophyte is meant that the endophyte is removed from its original environment (e.g. the natural environment if it is naturally occurring; the plant). For example, a naturally occurring endophyte present in nature in a living plant is not isolated, but the same endophyte separated from some or all of the coexisting materials in the natural environment, is isolated.

A plant of the Cannabaceae family includes plant parts thereof and may also be known as a Cannabis plant or a hemp plant.

In a preferred embodiment, the plant of the Cannabaceae family from which a Pseudomonas sp. endophyte is substantially purified or isolated is a Cannabis sativa, Cannabis indica or Cannabis ruderalis species plant, more preferably C. sativa. In particularly preferred embodiments, the plant of the Cannabaceae family is a C. sativa line denoted CannBio 2, CannBio 3, CannBio 4 and/or CannBio 5. A description of these plant lines is available in Australian Plant Breeder's Right (PBR) Application Nos. 2017/253, 2017/254, 2017/255 and 2017/256, respectively.

The Pseudomonas sp. endophyte may be substantially purified or isolated from any particular part of the plant, e.g. an organ. In preferred embodiments, the endophyte is substantially purified or isolated from a flower, flower bract, leaf, petiole, stem or root of the plant, more preferably a root.

The present invention arises from the discovery of Pseudomonas sp. endophyte strains of plants of the Cannabaceae family and their ability to form mutually beneficial relationships with plants, that may be used to confer certain benefits to plants. In particular, the present invention arises further from the surprising discovery that said endophytes may be bioprotective—and that certain species or strains of the Pseudomonas genus are particularly bioprotective—and may be used by application to plants to confer a bioprotection phenotype to a plant or part thereof, including for example resistance to pathogenic organisms.

Generally speaking, a bioprotective endophyte possesses genetic and/or metabolic characteristics that result in a bioprotection phenotype in a plant harbouring, or otherwise associated with, the endophyte. The bioprotection phenotype may include improved resistance to pests and/or diseases, enhanced biotic stress tolerance, reduced toxicity and/or enhanced vigour in the plant with which the endophyte is associated, relative to a plant not associated with the endophyte, or instead associated with a control endophyte such as a Paenibacillus pabuli bacterial strain. The pests and/or diseases may include, but are not limited to, bacterial and/or fungal pathogens.

In a preferred embodiment, the endophyte is capable of conferring a bioprotection phenotype to the plant or part thereof from which it is substantially purified or isolated, and/or is capable of conferring a bioprotection phenotype to a plant or part thereof to which it is inoculated. For clarity, an endophyte which presents with in vitro bioprotectant activity, for example an antifungal activity, may be taken to be capable of conferring the associated phenotype to a plant, for example resistance to that fungal pathogen, when said plant is infected, whether naturally or inoculated, with said pathogenic fungi.

By “inoculated” is meant to be placed in association with a plant to form a mutually beneficial relationship with the plant, whether that be on, in, or otherwise in close proximity to the plant. In preferred embodiments, the plant or part thereof to which the endophyte is inoculated is first free of that endophyte. In preferred embodiments, the inoculation method may be selected from the group consisting of spray inoculation, drop inoculation, co-inoculation, and coating.

In preferred embodiments, the bioprotection phenotype is improved resistance to pests and/or diseases, and preferably wherein the pests and/or diseases are bacterial and/or fungal pathogens. In preferred embodiments, the bioprotection phenotype is improved resistance to fungal pathogens, and in particular to one or both of a Botrytis sp. fungus and a Fusarium sp. fungus, preferably a Botrytis cinerea and/or a Fusarium proliferatum species strain.

The improved resistance which in these embodiments the endophyte is capable of conferring may generally be considered as compared to the resistance, or lack thereof as the case may be, of a plant or part thereof that is absent of the endophyte (“no endophyte control”), and/or as compared to the resistance of a plant that contains a Paenibacillus pabuli species bacterial strain, which again may be taken from a presented in vitro bioprotectant activity, or an observed in planta bioprotectant activity. The resistance to pathogens may be thought of as applied to a scale between complete resistance (i.e. 100% resistance, or say a pathogen infection score of 0), and no resistance (i.e. 0% resistance, or say a pathogen infection score of 5), where complete resistance may be indicated by no visible sign of infection over the normal course of otherwise unhindered pathogen growth or proliferation, and where no resistance may be indicated by visible sign of infection approximately equal to the normal course of unhindered pathogen growth or proliferation (i.e. as against no-endophyte control).

In preferred embodiments, the endophyte presents with in vitro resistance to a pathogenic Botrytis cinerea species strain of at least about 50%, preferably at least about 65% and more preferably of at least about 75%, against no-endophyte control, and/or at least about 10%, preferably about 15% and more preferably about 20%, greater resistance as compared to a Paenibacillus pabuli species bacterial strain. In preferred embodiments, the endophyte presents with in vitro resistance to a pathogenic Fusarium proliferatum species strain of at least about 30%, preferably at least about 40% and more preferably of at least about 45%, against no-endophyte control, and/or at least about 15%, preferably about 25% and more preferably about 30%, greater resistance as compared to a Paenibacillus pabuli species bacterial strain. Further, in preferred embodiments, the endophyte confers in planta resistance to a pathogenic Botrytis cinerea species strain of a pathogen infection score of at most about 3, preferably of at most about 2.5 and more preferably of at most about 2.2, against no-endophyte control.

The said plant or part thereof to which the endophyte is capable of conferring a bioprotection phenotype may be for example any plant which is or may be infected by a Botrytis sp. fungus and/or a Fusarium sp. fungus, say a Botrytis cinerea and/or a Fusarium proliferatum species strain. This includes plants of many important berry, medicinal and horticultural crops, such as plants of the Vitis, Cannabaceae, Fragaria, Rubus, Vaccinium, Ribes, Solanum, Brassica, Phaseolus and Lactuca families. In preferred embodiments, the said plant or part thereof to which the endophyte is capable of conferring a bioprotection phenotype is a plant of the Vitis family and preferably a plant of the Vitis vinifera species, and/or a plant of the Cannabaceae family and preferably a plant of the Cannabis sativa species. For Cannabaceae family plants and medicinal crops in particular, the bioprotectant microbes described herein may be amongst the only options available for the control of these Botrytis and/or Fusarium sp. pathogens.

In preferred embodiments, the Pseudomonas sp. endophyte is a Pseudomonas corrugata species strain, preferably a strain denoted EB-010, EB-013, EB-017 and/or EB-117 as deposited with The National Measurement Institute of 1/153 Bertie St, Port Melbourne, Victoria 3207 Australia on 24 Nov. 2020 with accession numbers V20/025722, V20/025723, V20/025725 and V20/025727, respectively.

Accordingly, in another aspect, the present invention provides a substantially purified or isolated bioprotective endophyte selected from the group consisting of EB-010, EB-013, EB-017 and EB-117 as deposited with The National Measurement Institute of 1/153 Bertie St, Port Melbourne, Victoria 3207 Australia on 24 Nov. 2020 with accession numbers V20/025722, V20/025723, V20/025725 and V20/025727, respectively.

All endophyte strains denoted EB-010, EB-013, EB-017 and EB-117 were substantially purified or isolated from the roots of a CannBio line of a C. sativa plant. All named endophytes are bioprotective, with the ability to confer one or more bioprotective properties to a plant—at least a plant of the Cannabaceae family (e.g. C. sativa) and/or Vitis family (e.g. V. vinifera)—at least the bioprotection phenotype of improved resistance to fungal pathogens, and in particular to Botrytis sp. fungus (e.g. Botrytis cinerea) and a Fusarium sp. fungus (e.g. Fusarium proliferatum). In particular, the named endophytes present with in vitro resistance to a pathogenic Botrytis cinerea species strain of at least about 75% against no-endophyte control, and of at least about 20% greater than a Paenibacillus pabuli species bacterial strain. They also present with in vitro resistance to a pathogenic Fusarium proliferatum species strain of at least about 45% against no-endophyte control, and of at least about 30% greater than a Paenibacillus pabuli species bacterial strain. The named endophytes also confers in planta resistance to a plant of the Vitis vinifera species to a pathogenic Botrytis cinerea species strain of a pathogen infection score at most about 2.2, against no-endophyte control.

Without wishing to be limited by theory, it is thought that the Pseudomonas sp. endophyte strains of plants of the Cannabaceae family, and especially the named endophyte, have the ability to confer a bioprotection phenotype, and in particular resistance to pests and/or diseases, at least in part by virtue of the production of a bioprotectant compound in the plant with which it is associated, and in particular a compound that is toxic to bacterial and/or fungal pathogens. Accordingly, the Pseudomonas sp. endophyte strains of plants of the Cannabaceae family that produce a bioprotectant compound in the plant generally an associated secondary-metabolite biosynthesis gene or gene cluster.

In preferred embodiments, the endophytes as described herein are accordingly positive for a secondary-metabolite biosynthesis gene cluster associated with a capability of the endophyte to confer on a plant a bioprotection phenotype as herein described, and in particular associated with a capability of the endophyte to produce a bioprotectant compound in the plant with which it is associated. Representative biosynthesis gene clusters include one or more of the fragin, arylpolyene (APE), bacteriocin, NAGGN, betalactone, cupriachelin, non-ribosomal peptide (NRP) siderophore, T1PKS (e.g. entolysin), siderophor, hserlactone, pyoverdin, crochelin and NRP+polyketide biosynthesis gene clusters. Preferably, the endophyte is positive for at least one of these biosynthesis gene clusters. In more preferred embodiments, the endophyte is positive for one or more, and preferably all, of the APE Vf (arylpolyene), fengycin (betalactone) and syringomycin (hserlactone) biosynthesis gene clusters. These biosynthesis gene clusters in particular, and especially the syringomycin biosynthesis gene cluster, may indicate that the endophyte is capable of producing a bioprotectant compound in a plant with which it is associated, and specifically one that is toxic to fungal pathogens, to confer on the plant the bioprotectant phenotype of improved resistance to fungal pathogens. All strains denoted EB-010, EB-013, EB-017 and EB-117 are positive for all of these biosynthesis gene clusters.

Similarly, the endophytes as described herein may be negative for one or more secondary metabolite biosynthesis gene clusters commonly associated with human toxicity, for example the pyocyanin gene cluster. This applies to at least the strains denoted EB-010, EB-013, EB-017 and/or EB-117; all are negative for the pyocyanin gene cluster.

Determining if an endophyte is positive or negative for a biosynthesis gene cluster will generally involve a genetic analysis, meaning an analysis of the nuclear and/or mitochondrial genetic nucleotide sequences—DNA and/or RNA—of the endophyte. By “positive” for a gene cluster is meant that the result of a genetic analysis indicates that the gene cluster is present in the endophyte. The genetic analysis may include comparing an endophyte nucleotide sequence to the nucleotide sequence of a known secondary metabolite biosynthesis gene cluster or a database thereof, for example antiSMASH. A positive result may be obtained by a sequence identity that is above a predetermined threshold, for example a sequence identity of about 100%, or for example of at least about 80%, 60%, 40% or 20%, or even 10%. A person skilled in the art will be familiar with sequence comparisons, appropriate sequence similarity thresholds and available databases. Similarly, by “negative” for a gene cluster is meant that the result of a genetic analysis of the endophyte indicates that the gene cluster is absent. A negative result may be other than a positive result. All strains denoted EB-010, EB-013, EB-017 and EB-117 are at least about 40% positive for the arylpolyene biosynthesis gene clusters, at least about 10% positive for the fengycin biosynthesis gene clusters and about 100% positive for the syringomycin biosynthesis gene cluster, using antiSMASH.

Similarly, all strains denoted EB-010, EB-013, EB-017 and EB-117 are negative for secondary metabolite biosynthesis gene clusters commonly associated with human toxicity, an in particular the pyocyanin gene cluster, again using antiSMASH.

In another aspect, the present invention provides a plant or part thereof inoculated with one or more Pseudomonas sp. endophytes as herein described.

The endophytes of the present invention may have the ability to be transferred through propagative material from one plant generation to the next. The endophyte may then spread or locate to other tissues as the plant grows, i.e. to roots. Alternatively, or in addition, the endophyte may be recruited to the plant root, e.g. from soil, and spread or locate to other tissues. In either sense, the endophyte may be said to be stably inoculated/infected to the plant.

Therefore, the present invention also provides a plant, plant propagative material or other plant part derived from a plant inoculated with an endophyte as herein described and infected therewith.

The present invention provides the use of an endophyte as herein described to produce a plant or part thereof infected, preferably stably infected, with said one or more of said endophytes.

The present invention also provides a method for conferring a bioprotection phenotype to a plant or part thereof, said method including inoculating to the plant or part thereof an endophyte as herein described. In all preferred embodiments, the plant or plant part inoculated or otherwise infected with an endophyte as herein described will exhibit an endophyte-conferred bioprotection phenotype, or in other words, the endophyte will confer thereto a bioprotection endophyte.

In a preferred embodiment of this aspect of the present invention, the plant or part thereof may be free of said endophyte prior to inoculation and may be stably infected with said endophyte.

In a preferred embodiment the method for conferring a bioprotection phenotype to a plant or part thereof includes the steps of:
a. substantially purifying or isolating one or more endophytes; and
b. inoculating the plant or part thereof with the isolated endophyte(s).

In a preferred embodiment, the method for conferring a bioprotection phenotype to a plant or part thereof includes subjecting said one or more isolated endophytes to one or more of:
i. an in vitro bioprotection activity assay;
ii. an in planta bioprotection activity assay; and
iii. an analysis for a secondary metabolite biosynthesis gene cluster
associated with the production of biocidal compounds, and based thereon, selecting one or more endophytes which are capable of conferring a bioprotection phenotype to the plant from which it is substantially purified or isolated and/or is capable of conferring a bioprotection phenotype to a plant or part thereof to which it is inoculated, wherein the plant or part thereof is inoculated with the selected endophyte.

Plants are often associated with many endophyte species and strains with varying functions and properties. The present invention also provides an efficient method for selecting in particular a bioprotective endophyte of a plant of the Cannabaceae family.

Thus, in another aspect, the present invention provides a method for selecting a bioprotective endophyte of a plant of the Cannabaceae family, said method comprising:
a. substantially purifying or isolating one or more endophytes; and
b. subjecting said one or more endophytes to one or more of:
   i. an in vitro bioprotection activity assay;
   ii. an in planta bioprotection activity assay; and
   iii. an analysis for a secondary metabolite biosynthesis gene cluster associated with the production of biocidal compounds, and based thereon, selecting an endophyte which is capable of conferring a bioprotection phenotype to the plant from which it is substantially purified or isolated and/or is capable of conferring a bioprotection phenotype to a plant or part thereof to which it is inoculated.

In this aspect, the plant of the Cannabaceae family, the secondary metabolite biosynthesis gene cluster and the plant or part thereof to which it the endophyte is capable of conferring a bioprotection phenotype may be as herein described. The skilled worker will further be familiar with techniques used to perform in vitro bioprotection activity assays and in planta bioprotection activity assays. The skilled worker will also be familiar with methods for substantially purifying or isolating endophytes, which generally includes:
a. providing one or more samples of said plant or part thereof;
b. preparing an extract(s) from said sample(s); and
c. growing endophyte colonies from said extract(s),
the colonies generally representing purified or isolated endophytes.

In a preferred embodiment of this aspect of the present invention, the step of substantially purifying or isolating one or more endophytes may include providing one or more samples of said plant or part thereof, preparing an extract(s) from said sample(s), and growing bacterial colonies from said extract(s).

In a preferred embodiment, the sample of plant material may be selected from one or more of the group consisting of flowers, flower bracts, leaves, petioles, roots and stem Preferably, the endophytes will be substantially purified or isolated from association with the roots of a plant of the Cannabaceae family, as this is where the largest population of endophytes reside.

In preferred embodiments, said method further includes the step of subjecting said selected endophyte(s) to genetic analysis to identify the endophyte species, and preferably the selected endophyte is a Pseudomonas sp. endophyte as herein described.

In this specification, the term ‘comprises’ and its variants are not intended to exclude the presence of other integers, components or steps.

In this specification, reference to any prior art in the specification is not and should not be taken as an acknowledgement or any form of suggestion that this prior art forms part of the common general knowledge in Australia or any other jurisdiction or that this prior art could reasonably expected to be combined by a person skilled in the art.

The present invention will now be more fully described with reference to the accompanying Examples and drawings. It should be understood, however, that the description following is illustrative only and should not be taken in any way as a restriction on the generality of the invention described above.

### BRIEF DESCRIPTION OF THE FIGURES

FIG. 1 depicts the 16S amplicon sequence of novel bacterial strain EB-010 (SEQ ID NO: 1).

FIG. 2 depicts the 16S amplicon sequence of novel bacterial strain EB-117 (SEQ ID NO: 2).

FIG. 3 depicts the 16S amplicon sequence of novel bacterial strain EB-017 (SEQ ID NO: 3).

FIG. 4 depicts the 16S amplicon sequence of novel bacterial strain EB-013 (SEQ ID NO: 4).

FIG. 5 depicts a phylogenetic tree based on an Average Nucleotide Identity (ANI) analysis of novel bacterial strains EB-117, EB-017, EB-013 and EB-010 and genome sequences of P. corrugata isolates from NCBI.

FIG. 6 depicts a phylogenetic tree based on Pan-genome analysis (Roary) of novel bacterial strains EB-117, EB-017, EB-013 and EB-010 and genome sequences of P. corrugata isolates from NCBI.

FIG. 7 contains photographs of grapes used in in planta bioprotection assays and provides a pictorial representation of disease inhibition ratings of from 1 to 5 based on percent lesion size caused by Botrytis cinereal pathogen infection.

FIG. 8 is a microbiome profile presented as a bar graph plotting the distribution of novel bacterial strain EB-010 in a mature medicinal Cannabis plant (F—Flower, FL—Flower Bract, O-L—Old Leaf, Y-L—Young Leaf, O-P—Old Petiole, Y-P—Young Petiole, STEM—Stem, R Root), established by mapping 16S metagenomic reads from these plant organs to the 16S sequence of EB-010.

### DETAILED DESCRIPTION OF EMBODIMENTS

In the following examples it is demonstrated that four novel Pseudomonas corrugata bacterial strains EB-117, EB-017, EB-013, EB-010 were isolated from medicinal Cannabis (Cannabis sativa) plants. They each display the ability to inhibit the growth of plant fungal pathogens in in vitro and in planta assays. The genomes of the four novel bacterial strains have been sequenced and are shown to be related to Pseudomonas corrugata. Analysis of the genome sequence has shown that all four bacterial strains have secondary metabolite gene clusters with known bioprotectant compounds, while they had no cluster associated with any human health effects.

### Example 1—Isolation of Bacterial Strains

Leaves, petioles, stems, flowers and roots were harvested from four different chemotypes (lines) (CannBio 2, 3, 4, 5) of mature Cannabis plants. Plants were grown in a greenhouse in pots containing two different substrates; standard potting mix and coconut matting/Jiffy. Root tissues were washed in sterile distilled water to remove soil particles and all the harvested tissues were cut into approximately 1 cm² pieces. The plant tissues and organs belonging to different Cannbio lines were separately placed in micro collection tubes and submerged in sufficient Phosphate Buffered Saline (PBS) to completely cover the plant tissue. Plant tissues were ground using a Qiagen TissueLyser II, for 1 minute at 30 Hertz. A 10 μl aliquot of the macerate was added to 90 μl of PBS. Subsequent 1 in 10 dilutions of the 10-suspension were used to create additional 10⁻² to 10⁻⁴ suspensions. Once the suspensions were well mixed, 50 μl aliquots of each suspension were plated onto Reasoners 2 Agar (R2A) for growth of bacteria. Dilutions that provided a good separation of bacterial colonies were subsequently used for isolation of individual bacterial colonies through re-streaking of single bacterial colonies from the dilution plates onto single R2A plates to establish a pure bacterial colony. Around 126 bacterial strains were obtained from mature plants grown in standard potting mix.

The novel bacterial strains EB-010, EB-013, EB-017 and EB-117 were collected from roots of medicinal Cannabis plants Cannbio 4, Cannbio 3, Cannbio 2 and Cannbio 5, respectively.

### Example 2—Identification of Novel Bacterial Strains

Proteomics

Bacterial isolates and the MALDI-TOF quality control Escherichia coli 25992 strain (from −80° C. storage) were streaked on to R2A agar plates and incubated at 22° C. for 48 hours in the dark. A single colony was picked from each culture and streaked on to a fresh R2A plate and incubated at 22° C. for an additional 48 hours in the dark.

The MALDI BioTyper® (Bruker Daltonics) system was used in initial bacterial identification. A standard dried droplet method was followed for sample preparation for MALDI-TOF analysis of all bacterial cultures. To provide biological and analytical replicates, n=2 single colonies were spotted twice for each isolate and a total of three MALDI-TOF runs were performed with freshly prepared 48 hour-old cultures to provide technical replicates. Similarly, the Bruker bacterial test standard (BTS) was spotted (1 μL aliquot each) as replicates for MALDI-TOF instrument calibration purposes.

Individual mass spectra profiles of all samples were acquired on a Bruker® MALDI-TOF mass spectrophotometer with flexControl v 3.3 software using the standard MBT-AutoX method. The equipment was calibrated using the BTS before every sample analysis, which comprised of eight calibration points and covered a mass range of between 1960 to 20,000 Da. The generated spectra were compared to a reference library, as well as the standard database supplied by Bruker using standard software. Based on the analogy between observed and referenced data, a score value was produced. Isolates with a cut-off score value of 22.0 were identified to the species level and isolates with a cut-off score of 22.3 were identified to the species level with significant accuracy.

The cut-off score values and initial species identification for the four novel bacterial isolates are provided in Table 1.

TABLE 1
Identification of novel bacterial strains using MALDI-TOF MS

| Isolate ID | Organism (Best Match) | Score |
|---|---|---|
| EB-010 | Pseudomonas corrugata | 2.379 |
| EB-013 | Pseudomonas corrugata | 2.431 |
| EB-017 | Pseudomonas corrugata | 2.473 |
| EB-117 | Pseudomonas chlororaphis | 2.106 |

Genomics

The genomes of novel bacterial strains EB-117, EB-017, EB-013, EB-010 were sequenced. These novel bacterial strains were retrieved from the glycerol collection stored at −80° C. by streaking on NA plates. Single colonies from these plates were grown overnight in Nutrient Broth and pelleted. These pellets were used for genomic DNA extraction using the bacteria protocol of Wizard® Genomic DNA Purification Kit (A1120, Promega). To enable full genome assembly, long reads were generated for the four novel bacterial strains by sequencing DNA using Oxford Nanopore Technologies (ONT) MinION platform. The DNA from the Wizard® Genomic DNA Purification Kit was first assessed with the genomic assay on Agilent 2200 TapeStation system (Agilent Technologies, Santa Clara, CA, USA) for integrity (average molecular weight 230 Kb). The sequencing library was prepared using an in-house protocol modified from the official protocols for transposases-based library preparation kits (SQK-RAD004/SQK-RBK004, ONT, Oxford, UK). All libraries were sequenced on a MinION Mk1B platform (MIN-101B) with R9.4 flow cells (FLO-MIN106) and under the control of MinKNOW software. After the sequencing run finished, the fast5 files that contain raw read signals were transferred to a separate, high performance computing Linux server for local base-calling using ONT's Albacore software (Version 2.3.1) with default parameters. The sequencing summary file produced by Albacore was processed by the R script minion qc (https://github.com/roblanf/minion_qc) and NanoPlot (De Coster et al. 2018) to assess the quality of the sequencing run, while Porechop (Version 0.2.3, https://github.com/rrwick/Porechop) was used to remove adapter sequences from the reads. Reads that were shorter than 300 bp were removed and the worst 5% of reads (based on quality) were discarded using Filtlong (Version 0.2.0, https://github.com/rrwick/Filtlong).

The whole genome sequence of the four novel bacterial strains were assembled using Unicycler (Wick et al. 2017). MinION reads were mainly used to resolve repeat regions in the genome. Multiple rounds of Racon (Vaser et al. 2017) polishing were then carried out to generate consensus sequences. Assembly graphs were visualised by using Bandage (Wick et al. 2015).

A complete circular chromosome sequence was produced for the four novel bacterial strains. The genome size for the novel bacterial strains EB-117, EB-017, EB-013, EB-010 were 6,624,132 bp, 6,657,479 bp, 6,539,460 bp and 6,487,017 bp, respectively (Table 2).

TABLE 2
Summary of properties of the final genome sequence assembly

| | Genome | Plasmid | GC content | |
|---|---|---|---|---|
| Strain ID | size (bp) | Size (bp) | (%) | Coverage |
| EB-117 | 6,624,132 | 135,326 | 60.1 | 385.0× |
| EB-017 | 6,657,479 | 15,831 | 60.3 | 604.7× |
| EB-013 | 6,539,460 | | 60.3 | 604.9× |
| EB-010 | 6,487,017 | 20,489 | 60.4 | 177.9× |

Novel bacterial strains EB-117, EB-017 and EB-010 also contained a plasmid, ranging in size from 15,831 bp to 135,326 bp. The percent GC content was around 60%. The novel bacterial strains were annotated by Prokka (Seemann 2014) with a custom, genus-specific protein database to predict genes and corresponding functions, which were then screened manually to identify specific traits. The number of genes for the novel bacterial strains EB-117, EB-017, EB-013, EB-010 were 6090, 7768, 7528, and 5829 genes, respectively (Table 3).

TABLE 3
Summary of genome coding regions

| | No. of | No. of | No. of | | No. of |
|---|---|---|---|---|---|
| Strain ID | tRNA | tmRNA | rRNA | No. of CDS | genes |
| EB-117 | 69 | 1 | 16 | 6004 | 6090 |
| EB-017 | 72 | 1 | 17 | 7678 | 7768 |
| EB-013 | 70 | 1 | 16 | 7441 | 7528 |
| EB-010 | 70 | 1 | 16 | 5742 | 5829 |

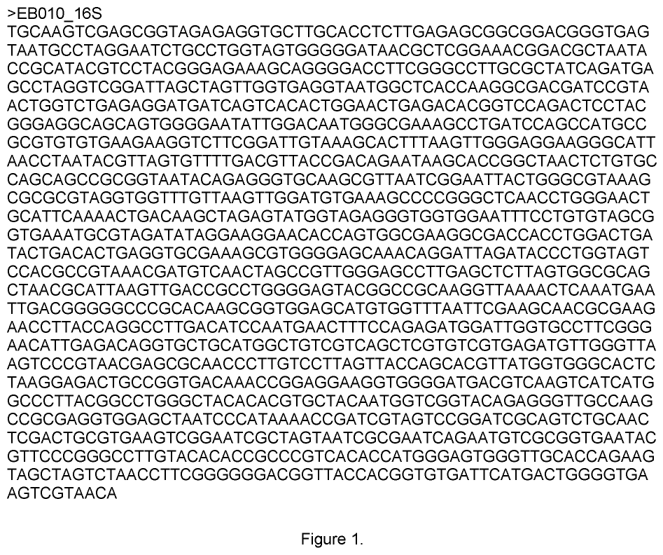

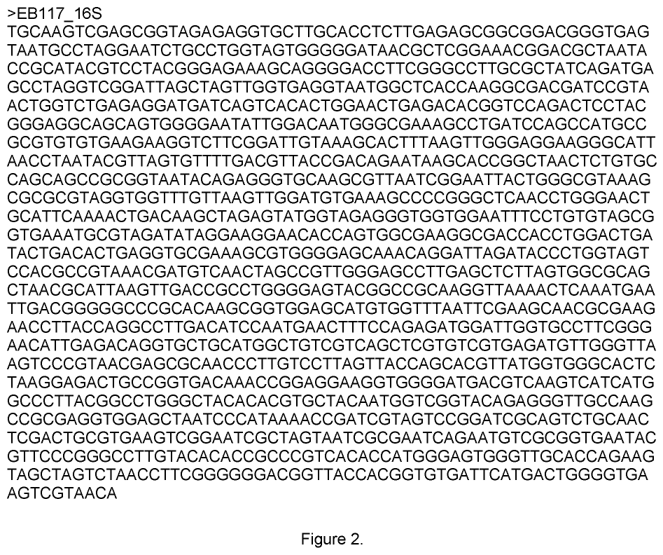

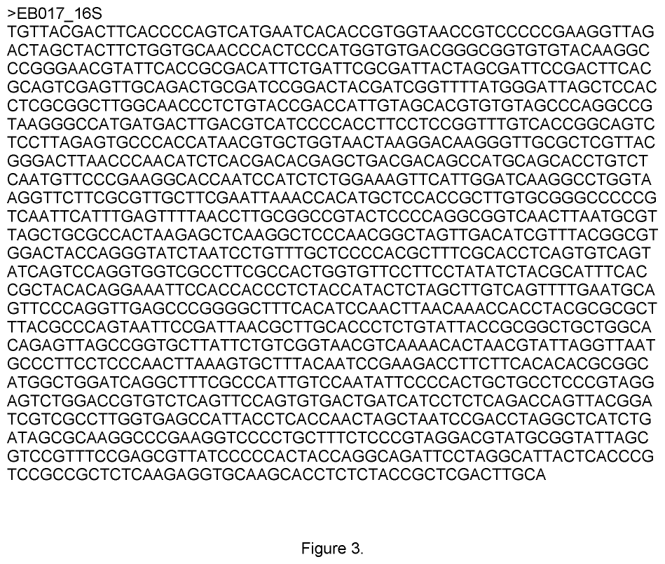

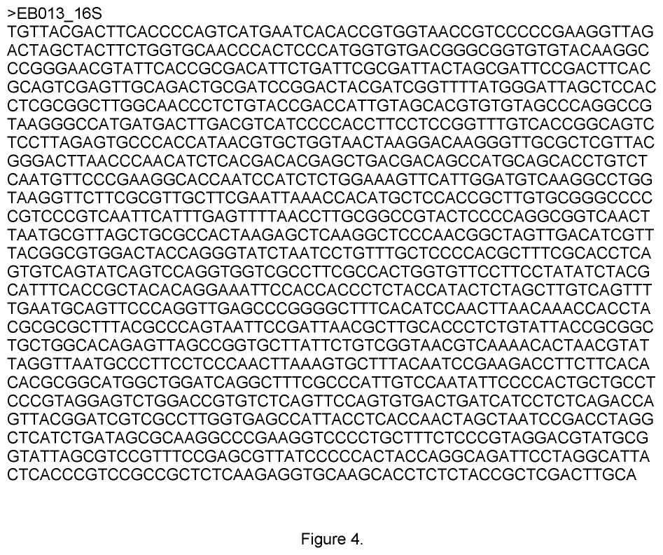

A phylogenetic analysis of the novel bacterial strains EB-117, EB-017, EB-013, EB-010 was undertaken by sequence homology comparison of the 16S rRNA gene regions extracted from whole genome sequence of each bacteria (FIGS. 1-4). The sequences were aligned by BLASTn on NCBI against the non-redundant nucleotide database and the 16S ribosomal RNA database. The preliminary taxonomic identification of the novel bacterial strains EB-117, EB-017, EB-013, EB-010 based on 16S sequences were Pseudomonas corrugata (Tables 4 and 5)

TABLE 4
BLASTn hit against database nr; Pseudomonas corrugata 16S ribosomal
RNA gene, partial sequence

| Strain | Query | E- | % | | |
|---|---|---|---|---|---|
| ID | Coverage | Value | Identity | Species | Accession |
| EB-117 | 100% | 0 | 100% | Pseudomonas | NR117826.1 |
| | | | | corrugata | |
| EB-017 | 100% | 0 | 99.79% | Pseudomonas | NR117826.1 |
| | | | | corrugata | |
| EB-013 | 100% | 0 | 99.59% | Pseudomonas | NR117826.1 |
| | | | | corrugata | |
| EB-010 | 100% | 0 | 100% | Pseudomonas | NR117826.1 |
| | | | | corrugata | |

TABLE 5
BLASTn hit against database 16S ribosomal RNA; Pseudomonas
corrugata strain BS3649 16S ribosomal RNA gene, genome assembly

| Strain | Query | E- | % | | |
|---|---|---|---|---|---|
| ID | Coverage | Value | Identity | Species | Accession |
| EB- | 100% | 0 | 99.96% | Pseudomonas corrugata | LT629798.1 |
| 117 | | | | | |
| EB- | 100% | 0 | 99.76% | Pseudomonas corrugata | LT629798.1 |
| 017 | | | | | |
| EB- | 100% | 0 | 99.72% | Pseudomonas corrugata | LT629798.1 |
| 013 | | | | | |
| EB- | 100% | 0 | 99.94% | Pseudomonas corrugata | LT629798.1 |
| 010 | | | | | |

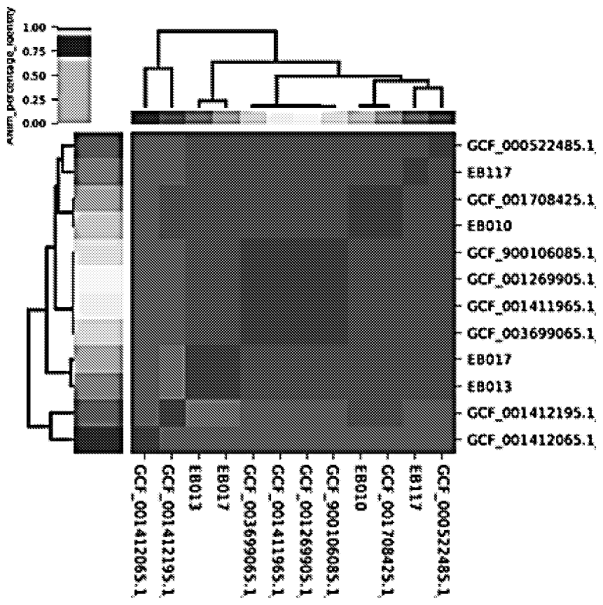

Six P. corrugata genome sequences and one P. chlororaphis genome sequence that were publicly available on NCBI were acquired and used for average nucleotide identity (ANI, no P. chlororaphis strain) calculation and pan-genome/comparative genome sequence analysis alongside P. corrugata novel bacterial strains EB-117, EB-017, EB-013 and EB-010. ANI values were calculated using the Pyani package and a phylogenetic tree was generated. (Pritchard L. 2016). In the phylogenetic tree EB-013 and EB-017 formed a clade, while EB-117 and GCF_000522485.1 formed a clade, and EB-010 and GCF_001708425.1 formed a clade (FIG. 5). The four novel bacterial strains EB-117, EB-017, EB-013 and EB-010 had >99% average nucleotide identity to each other and P. corrugata isolates. Based on an ANI species cut of 95-96%, it is evident that isolates EB-117, EB-017, EB-013 and EB-010 are representative isolates of P. corrugata.

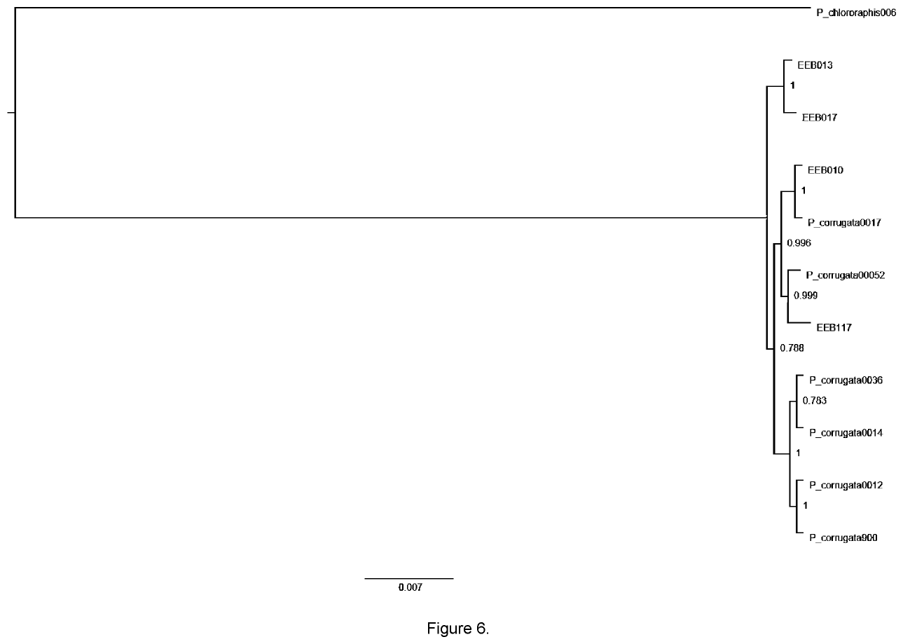

Prokka (Seemann 2014) annotated novel bacterial genomes were provided to Roary (Page et al. 2015) and a total of 625 genes that are shared by all eleven strains were identified. PRANK (Löytynoja 2014) was then used to perform a codon aware alignment and visualization of phylogenetic tree derived from core gene alignment was produced with FigTree v1.4.4 (Rambaut A. 2018). The novel bacterial strains EB-117, EB-017, EB-013 and EB-010 clustered together with, P. corrugata reference isolates, and separate from the P. chlororaphis strain, confirming these novel bacterial strains belong to the species P. corrugata (FIG. 6).

### Example 3—Bioprotection Activity (In Vitro) of Novel Bacterial Strains

In vitro bioassays were established to test the bioactivity of the novel bacterial strains EB-010, EB-013, EB-017 and EB-117 against two major fungal pathogens of medicinal Cannabis Botrytis cinerea (VPRI 42964) and Fusarium proliferatum (VPRI 42958). An unrelated bacterial strain X (Paenibacillus pabuli) was used as a negative control. The two fungal pathogens were isolated from infected medicinal Cannabis plants obtained from the National Collection of Fungi (Herbarium VPRI) and the Agriculture Victoria Research collection. Bacterial isolates were tested for in vitro antagonism towards fungal pathogens by following the standard co-inoculation technique with three inoculation methods (dual culture one species streak, dual culture mix species streak and four spot drop-inoculation) on NA plates (BD Biosciences). For both the dual culture one species streak and dual culture mix species streak, a 6 mm×6 mm agar plug of actively growing mycelia from the pathogen was placed at the centre of one side of the plate. Nutrient agar plates were incubated for 24-48 hours or until the fungi started establishing on the plate at 24° C. in the dark. Once the fungal culture established in NA plate each bacterial strain was freshly streaked on opposite side of the same plate in such a way that smear of bacterial colony completely cover the half of the plate, and the plate was then incubated at 26° C. for 7-14 days in the dark.

For the four-spot drop-inoculation method each bacterial strain was drop-inoculated (20 μL) onto four equidistant points on a Nutrient Agar (BD Biosciences) plate, which was then incubated overnight at 28° C. A 6 mm×6 mm agar plug of actively growing mycelia from the pathogen was placed at the centre of the plate. All the bioassays were incubated for 7-14 days at 28° C. in the dark, and then the diameter of the fungal colony on the plate was recorded. For each treatment three plates were prepared as biological triplicates. OriginPro 2018 (Version b9.5.1.195) was used to carry out One-way ANOVA and Tukey Test to detect the presence of any significant difference (p≤0.05) between treatments.

The four novel bacterial strains inhibited the growth of both pathogens, indicating that they have broad spectrum biocidal activity, unlike strain X. Novel bacterial strain EB-017 significantly inhibited the growth of Botrytis cinerea with the highest percentage inhibition (82.30%) in comparison to strain X (Paenibacillus pabuli) (58.33%). Novel bacterial strains EB-010, EB-013 and EB-117 also significantly inhibited the growth of Botrytis cinerea in comparison to strain X (Paenibacillus pabuli) (82.20%, 79.50% and 78.33%, respectively cf. 58.33% for control). Novel bacterial strain EB-010 significantly inhibited the growth of Fusarium proliferatum with highest percentage inhibition (53.5%) in comparison to strain X (Paenibacillus pabuli) (16.3%). Novel bacterial strains EB-013, EB-017 and EB-117 also significantly inhibited the growth of Fusarium proliferatum in comparison to strain X (Paenibacillus pabuli) (51.2%, 51.2% and 48.8%, respectively cf. 16.3% for control). These results are presented in Table 6 (superscript ‘^{a}’ represents statistical significance).

TABLE 6
Bioprotection bioassay indicating the percentage inhibition (versus the
control) of the Pseudomonas sp. novel bacterial strains EB-010, EB-013
and EB-017 against plant pathogenic fungi, Botrytis cinerea and Fusarium
proliferatum.

| Pathogen ID | EB-010 | EB-013 | EB-017 | EB-117 | strain X |
|---|---|---|---|---|---|
| Botrytis cinereal | 82.20^{a} | 79.50^{a} | 82.30^{a} | 78.33^{a} | 58.33^{b} |
| (VPRI 42964) | | | | | |
| Fusarium proliferatum | 53.5^{a} | 51.2^{a} | 51.2^{a} | 48.8^{a} | 16.3^{b} |
| (VPRI 42958) | | | | | |

### Example 4—Bioprotection Activity (in Planta) of Novel Bacterial Strains—Grapes

A table grape (Vitis vinifera) assay was established to evaluate the in planta bioprotection activity of novel bacterial strains EB-010, EB-013, EB-017 and EB-117 against the fungal phytopathogen Botrytis cinerea (VPRI 42964). The bacterial strains were cultured in nutrient broth (BD Bioscience) and incubated overnight at 28° C. in a shaking incubator (200 rpm). Next day, table grapes (ripe white seedless table grapes) were surface sterilised with 70% alcohol for 30 seconds, 1% sodium hypochlorite (NaOCl) for 1 minute, then rinsed with sterile distilled water (SDW) three times. Surface sterilised fruits were air dried inside the laminar floor or wiped with clean paper towels to remove the excess water.

Each bacterial strain was spray inoculated onto table grapes to coat the whole fruit. Six fruit were tested per bacterial isolate. Negative control fruit were coated with nutrient broth medium, and positive control fruit were left without inoculating the bacteria. The inoculated table grapes were kept on plastic trays elevated inside sealed moist incubation chambers, which consisted of moistened paper towelling lining the base of rectangular disposable food containers and incubated overnight at 26° C. in the dark.

To make conidial suspensions of the pathogenic fungus, 10 mL of SDW was added to 7-15 day-old cultures, the mycelia were scraped with a sterile glass rod and the suspension filtered through muslin cloth. The concentration of spore suspension was adjusted to 10⁶ conidia/mL. Bacteria-treated, overnight-incubated fruits were inoculated by adding 5 μL of the spore suspension from each isolate onto the upper surface of the fruit; or a 6 mm×6 mm agar plug of actively growing mycelia from the pathogen was placed at the centre of the fruit. All the fruits were inoculated with pathogen by both wound and non-wound methods. The wound method involved pricking the fruit surface with a sterilised needle after adding the spore suspension/mycelial plug. Control fruits were treated with 5 μL of SDW and the experiment was carried out seven times.

A range of strategies were evaluated to optimise the bioactivity of bacteria, to optimise the pathogen infection, and simulate commercial application.
Bioactive bacteria inoculation technique—spray, drop inoculation or coating (short/long duration)
Single or mixed cultures of bioactive bacteria
Pathogen inoculum source—spores or mycelia on agar
Wounding—with and without
Order of inoculation (pathogen—bioactive bacteria; bioactive bacteria—pathogen)
Condition of fruit (age/maturity)
Replication

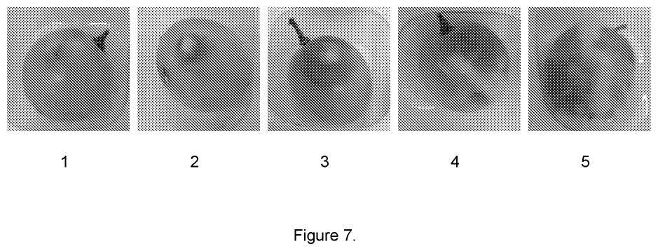

The bioprotection activity (inhibition against symptoms development) at the inoculation site were evaluated at 7-14 days after inoculation based on percent lesion size (the size of necrotic zones and the fungal hyphal growth). A disease inhibition rating was assigned to each from between the values of 0 to 5, where 1=strong disease inhibition, to 5=no disease inhibition (FIG. 7).

The results represent the mean disease inhibition score across all seven trials. Novel bacterial strains EB-010, EB-013 (individual) and the mix of EB-013, EB-017 and EB-117 (combinations of isolates) showed the highest in planta bioprotection activity against the fungal phytopathogen Botrytis cinerea (FIG. 7 and Table 7).

TABLE 7
Bioprotection bioassay indicating the mean inhibition scores
of the novel bacterial strains EB-010, EB-013, EB-017 and EB-
117 against plant pathogenic fungi, Botrytis cinerea.

| Sample name | Species | Mean | Activity rating |
|---|---|---|---|
| EB-017 | Pseudomonas corrugata | 1.67 | ** |
| EB-117 | Pseudomonas corrugata | 2.17 | * |
| EB-025 | Pseudomonas libanensis | 2.8 | * |
| EB-010 | Pseudomonas corrugata | 0.83 | *** |
| EB-013 | Pseudomonas corrugata | 0.3 | *** |
| EB-117, EB-010, EB- | Pseudomonas corrugata | 2.17 | * |
| 013 (mixture) | | | |
| EB-117, EB-017, EB- | Pseudomonas corrugata | 1.5 | ** |
| 013 (mixture) | | | |

### Example 5—Genome Sequence Features Supporting the Bioprotection Niche of the Novel Bacterial Strains

Secondary Metabolite Biosynthesis Gene Clusters

The genome sequences of the four novel bacterial strains EB-010, EB-013, EB-017 and EB-117 were assessed for the presence of features associated with bioprotection. The annotated genome sequences were analysed by antiSMASH (Weber et al. 2015) to identify secondary metabolite biosynthesis gene clusters that are commonly associated with the production of biocidal compounds that aid in their defence. Annotated genome sequences were passed through antiSMASH with the following options: —clusterblast—asf—knownclusterblast—subclusterblast—smcogs—full-hmmer. A total of three secondary metabolite gene clusters were identified in the genome sequences of all four novel bacterial strains (Table 8). A total of 11 biosynthetic gene clusters were identified, with novel bacterial strain EB-010 and EB-117 having 9 clusters, while EB-013 had 8 and EB-017 had 7. All isolates contained the cluster associated with syringomycin production, but had no cluster associated with any known human health effects.

TABLE 8
Secondary metabolite biosynthesis gene clusters in the novel bacterial strains identified using antiSMASH (Weber et al. 2015).

| | Type | Most similar known cluster | | EB-013 | Similarity | EB-010 | Similarity | EB-017 | Similarity | EB-117 | Similarity |
|---|---|---|---|---|---|---|---|---|---|---|---|
| 1 | NRPS-like | fragin | NRP | Y | 37% | Y | 37% | | | Y | 60% |
| 2 | arylpolyene | APE Vf | Other | Y | 40% | Y | 45% | Y | 45% | Y | 45% |
| 3 | bacteriocin | | | Y | | Y | | Y | | Y | |
| 4 | NAGGN | | | Y | | Y | | Y | | Y | |
| 5 | betalactone | fengycin | NRP | Y | 13% | Y | 13% | Y | 20% | Y | 13% |
| 6 | NRPS | cupriachelin | NRP:NRP | | | Y | 11% | | | | |
| | | | siderophore | | | | | | | | |
| 7 | NRPS-like, T1PKS | entolysin | NRP | | | Y | 17% | | | Y | 17% |
| 8 | siderophore | | | Y | | Y | | Y | 12% | Y | |
| 9 | hserlactone, NRPS | syringomycin | NRP | Y | 100% | Y | 100% | Y | 100% | Y | 100% |
| 10 | NPRS | pyoverdin | NRP | Y | 1% | | | | | | |
| 11 | NRPS | crochelin | NRP + Polyketide | | | | | Y | 11% | Y | 7% |
| | | | | 8 | | 9 | | 7 | | 9 | |

### Example 6—Distribution of Novel Bacterial Strains in Medicinal Cannabis Plants

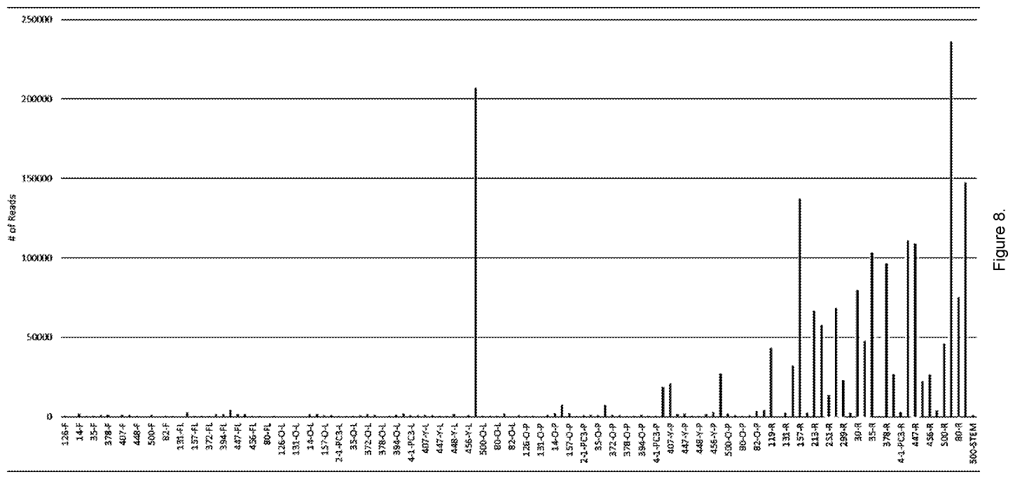

For microbiome profiling, flowers, flower bracts, leaves (old and young), petioles (old and young), roots and stem were collected from mature plants. DNA extraction was performed in 96-well plates using the QIAGEN MagAttract 96 DNA Plant Core Kit according to manufacturers' instructions with minor modifications for use with a Biomek FX liquid handling station. The bacterial microbiome was profiled targeting the V4 region (515F and 806R) of the 16S rRNA gene according to the Illumina 16S Metagenomic Sequencing Library Preparation protocol, with minor modifications to include the use of PNA PCR blockers to reduce amplification of 16S rRNA genes sequences derived from the plant chloroplast genome and mitochondrial genome (Wagner et al., 2016). Paired-end sequencing was performed on a MiSeq to generate 2×300 bp reads. Sequence data was trimmed and merged using PandaSEQ (removal of low quality reads, 8 bp overlap of read 1 and read 2, removal of primers, final merged read length of 253 bp) (Massela et al., 2012). Gydle software suite (https://www.gydle.com/) was used for dereplication, taxonomical assignment and removal of organelle OTUs. Reads were mapped to the 16S sequence of EB-010 as a representative of the four novel bacterial strains to determine the distribution of the strains through medicinal Cannabis plants. Reads were identified in all organs, with numbers ranging around 1000 for flowers, flower bracts, leaves (old and young), petioles (old and young) and stems, while numbers were higher in roots (up to 236,330) (FIG. 8). As such, the novel bacterial strains appear ubiquitously distributed across the medicinal Cannabis plant, but more concentrated in roots than other organs.

### Example 7—Bioprotection Activity (in Planta) of Novel Bacterial Strains—Medicinal Cannabis

A medicinal Cannabis (Cannabis sativa) detached leaf assay was established to evaluate the in planta bioprotection activity of novel bacterial strains EB-010, EB-013, EB-017 and EB-117 against the fungal phytopathogen Borytis cinerea (VPRI 42964). An additional isolate was also evaluated, Pseudomonas libanensis (EB-025). The bacterial strains were cultured in nutrient broth (BD Bioscience) and incubated overnight at 28° C. in a shaking incubator (200 rpm). The next day, young leaves harvested from medicinal Cannabis were surface sterilised with 70% ethanol for 30 seconds, 1% sodium hypochlorite (NaOCl) for 1 minute, then rinsed with sterile distilled water (SDW) three times. Surface sterilised leaves were air dried inside the laminar flow and wiped with sterile paper towels to remove the excess water.

Optical density (OD) measurements were taken for overnight cultures of each bacterial strain using a spectrophotometer (Eppendorf BioPhotometer D30), following which the OD600 value was adjusted to 1 by diluting with nutrient broth medium, if needed. Each bacterial strain was spray inoculated onto leaves to coat the whole leaf using a handheld atomiser. Three leaves were tested per bacterial isolate. Negative control leaves were coated with nutrient broth medium, and positive control leaves were untreated. The inoculated leaves were incubated overnight at 26° C. in the dark in a plastic box (used separate box per bacteria) with wet sterile paper towel, prior to the inoculation of the pathogen.

A conidial suspensions of the fungal pathogen B. cinerea was prepared by adding 10 mL of SDW to 15 day-old fungal cultures, following which mycelia were scraped from the culture with a sterile glass rod, and the suspension filtered through sterile muslin cloth. The concentration of the spore suspension was adjusted to 10⁶ conidia/mL. Leaves were inoculated by adding 5 μL of the spore suspension from each isolate onto the upper surface of the leaf. All leaves were wounded as the pathogen was inoculated by pricking the leaf surface with a sterile needle and then adding the spore suspension. Negative control leaves were treated with 5 μL of SDW.

The bioprotection activity (inhibition against fungal growth) was evaluated at 5 days after inoculation of the pathogen. An assessment of fungal growth was based on the size of necrotic zones and the aerial hyphal growth. A disease expression rating was assigned for each between the values of 1 to 5, where 1=no disease (no aerial hyphae, no necrosis), 2=minor disease (no aerial hyphae, necrosis), 3=moderate disease (moderate aerial hyphae, necrosis), 4=severe disease (severe aerial hyphae, necrosis) and 5=extensive disease (extensive aerial hyphae, necrosis). Statistical analysis was performed on disease inhibition rating using OriginPro. The four novel bacterial strains EB-010, EB-013, EB-017 and EB-117 significantly (p<0.05) reduced the growth of B. cinerea infection in detached Cannabis leaves, compared to the fungi only treatment (positive control) and P. libanensis (EB-025) (Table 9). Novel bacterial strains EB-013 was the most active at reducing the growth of B. cinerea, however all P. corrugata isolates were significantly equivalent.

TABLE 1
Bioprotection detached leaf bioassay indicating the mean disease expression
rating of the novel bacterial strains EB-010, EB-013, EB-017 and EB-117
against plant pathogenic fungi, Botrytis cinerea.

| | | Disease | |
|---|---|---|---|
| | | Expression | |
| Sample name | Species | Rating | Activity rating |
| EB-017 | Pseudomonas corrugata | 1.2 | *** |
| EB-117 | Pseudomonas corrugata | 1.2 | *** |
| EB-010 | Pseudomonas corrugata | 1.2 | *** |
| EB-013 | Pseudomonas corrugata | 1.0 | *** |
| EB-025 | Pseudomonas libanensis | 3 | * |
| Fungi only | NA | 3.8 | NA |

Finally, it is to be understood that various alterations, modifications and/or additions may be made without departing from the spirit of the present invention as outlined herein.

### REFERENCES

1. Chen, S., Y. Zhou, Y. Chen and J. Gu. 2018. fastp: an ultra-fast all-in-one FASTQ preprocessor. Bioinformatics 34: i884-i890. doi:10.1093/bioinformatics/bty560.
2. De Coster, W, D'Hert, S, Schultz, DT, Cruts, M & Van Broeckhoven, C 2018, ‘NanoPack: visualizing and processing long read sequencing data’, Bioinformatics.
3. Löytynoja, A 2014, ‘Phylogeny-aware alignment with PRANK’, in D J Russell (ed.), Multiple Sequence Alignment Methods, Humana Press, Totowa, NJ, pp. 155-70, DOI 10.1007/978-1-62703-646-7_10, <https://doi.org/10.1007/978-1-62703-646-7_10>.
4. Massela A P, Bartram A K, Truszkowski J M, Brown D G, Neufeld J D. 2012. ‘PANDAseq: PAired-eND Assembler for Illumina sequences’. BMC Bioinformatics 13(31).
5. Page, A J, Cummins, C A, Hunt, M, Wong, V K, Reuter, S, Holden, M T, Fookes, M, Falush, D, Keane, J A & Parkhill, J 2015, ‘Roary: rapid large-scale prokaryote pan genome analysis’, Bioinformatics, vol. 31, no. 22, pp. 3691-3.
6. Seemann, T 2014, ‘Prokka: rapid prokaryotic genome annotation’, Bioinformatics, vol. 30, no. 14, pp. 2068-9.
7. Vaser, R, Sovic, I, Nagarajan, N & Sikic, M 2017, ‘Fast and accurate de novo genome assembly from long uncorrected reads’, Genome Res, vol. 27, no. 5, pp. 737-46.
8. Wagner M R, Lundberg D S, Tijana G, Tringe S G, Dangl J L, Mitchell-Olds T. ‘Host genotype and age shape the leaf and root microbiomes of a wild perennial plant’. Nature Communications. 2016 Jul. 12; 7:12151.
9. Walker, B J, Abeel, T, Shea, T, Priest, M, Abouelliel, A, Sakthikumar, S, Cuomo, C A, Zeng, Q, Wortman, J, Young, S K & Earl, A M 2014, ‘Pilon: an integrated tool for comprehensive microbial variant detection and genome assembly improvement’, PLoS One, vol. 9, no. 11, p. e112963.
10. Weber, T, Blin, K, Duddela, S, Krug, D, Kim, H U, Bruccoleri, R, Lee, S Y, Fischbach, M A, Muller, R, Wohlleben, W, Breitling, R, Takano, E & Medema, M H 2015, ‘antiSMASH 3.0—a comprehensive resource for the genome mining of biosynthetic gene clusters’, Nucleic Acids Res, vol. 43, no. W1, pp. W237-43
11. Wick, R R, Schultz, M B, Zobel, J & Holt, K E 2015, ‘Bandage: interactive visualization of de novo genome assemblies’, Bioinformatics, vol. 31, no. 20, pp. 3350-2.
12. Wick, R R, Judd, L M, Gorrie, C L & Holt, K E 2017, ‘Unicycler: Resolving bacterial genome assemblies from short and long sequencing reads’, PLOS Computational Biology, vol. 13, no. 6, p. e1005595.

1-35. (canceled)

36. A substantially purified or isolated Pseudomonas sp. endophyte of a plant of the Cannabaceae family, wherein said endophyte is capable of conferring a bioprotection phenotype to the plant or part thereof from which it is substantially purified or isolated and/or is capable of conferring a bioprotection phenotype to a plant or part thereof to which it is inoculated.

37. An endophyte according to claim 36, wherein the bioprotection phenotype is improved resistance to pests and/or diseases.

38. An endophyte according to claim 37, wherein said disease is infection by a fungal pathogen.

39. An endophyte according to claim 38, wherein said fungal pathogen is a Botrytis sp. fungus or a Fusarium sp. fungus.

40. An endophyte according to claim 36, wherein one or more of the following applies:
i) said endophyte is capable of presenting with an in vitro resistance to a pathogenic Botrytis cinerea species strain of at least about 75% against no-endophyte control, and/or of at least about 20% greater resistance than a Paenibacillus pabuli species bacterial strain;
ii) said endophyte is capable of presenting with an in vitro resistance to a pathogenic Fusarium proliferatum species strain of at least about 45% against no-endophyte control, and/or of at least about 30% greater resistance than a Paenibacillus pabuli species bacterial strain; and
iii) said endophyte is positive for one or more of the fragin, arylpolyene (APE), bacteriocin, NAGGN, betalactone, cupriachelin, non-ribosomal peptide (NRP) siderophore, TIPKS (e.g. entolysin), siderophor, hserlactone, pyoverdin, crochelin and NRP+polyketide biosynthesis gene clusters.

41. An endophyte according to claim 40, wherein one or both of the following apply:
i) said endophyte is positive for one or more of the APE Vf (arylpolyene), fengycin (betalactone) and syringomycin (hserlactone) biosynthesis gene clusters; and
ii) said endophyte is positive for the syringomycin biosynthesis gene cluster.

42. An endophyte according to claim 36, wherein said endophyte is negative for one or more secondary metabolite biosynthesis gene clusters commonly associated with human toxicity.

43. An endophyte according to claim 42, wherein a secondary metabolite biosynthesis gene cluster commonly associated with human toxicity and for which the endophyte is negative is the pyocyanin gene cluster.

44. An endophyte according to claim 36, wherein said plant or part thereof to which it is capable of conferring a bioprotection phenotype is one which is or may be infected by a Botrytis sp. Fungus and/or a Fusarium sp. Fungus and wherein said plant or part thereof to which it is capable of conferring a bioprotection phenotype is of the Vitis, Cannabaceae, Fragaria, Rubus, Vaccinium, Ribes, Solanum, Brassica, Phaseolus and/or Lactuca families.

45. An endophyte according to claim 44, wherein said plant or part thereof to which it is capable of conferring a bioprotection phenotype is of the Vitis and/or Cannabaceae family.

46. An endophyte according to claim 36, wherein said endophyte is capable of conferring in planta resistance to a plant of the Vitis vinifera species to a pathogenic Botrytis cinerea species strain of a pathogen infection score at most about 2.2, against no-endophyte control.

47. An endophyte according to claim 36, wherein said plant of the Cannabaceae family is a Cannabis saliva, Cannabis indica or Cannabis ruderalis species plant.

48. An endophyte according to claim 36, wherein said endophyte is substantially purified or isolated from a flower, flower bract, leaf, petiole, stem or root of the plant of the Cannabaceae family.

49. An endophyte according to claim 48, wherein said endophytes is substantially purified or isolated from a root.

50. An endophyte according to claim 36, wherein said endophyte is a Pseudomonas corrugata species strain.

51. An endophyte according to claim 36, wherein said endophyte is a strain denoted EB-010, EB-013, EB-017 and/or EB-117 as deposited with The National Measurement Institute on 24 Nov. 2020 with accession numbers V20/025722, V20/025723, V20/025725 and V20/025727, respectively.

52. A plant or part thereof inoculated with one or more endophytes according to claim 36 and exhibiting a bioprotection phenotype conferred to the plant by the endophyte.

53. A method for conferring a bioprotection phenotype to a plant or part thereof, said method including inoculating the plant or part thereof with an endophyte according to claim 36.

54. A method for selecting a bioprotective endophyte of a plant of the Cannabaceae family, said method comprising:
a. substantially purifying or isolating one or more endophytes; and
b. subjecting said one or more endophytes to one or more of:
i. an in vitro bioprotection activity assay;
ii. an in planta bioprotection activity assay; and
iii. an analysis for a secondary metabolite biosynthesis gene cluster associated with the production of biocidal compounds, and
based thereon, selecting an endophyte which is capable of conferring a bioprotection phenotype to the plant from which it is substantially purified or isolated and/or is capable of conferring a bioprotection phenotype to a plant or part thereof to which it is inoculated and wherein a selected endophyte is a Pseudomonas sp. endophyte.

55. A method according to claim 54, further including subjecting said endophyte colonies or selected endophyte(s) to genetic analysis to identify the endophyte species.

56. A method according to claim 54, wherein a selected endophyte is substantially purified or isolated from the roots a plant of the Cannabaceae family.
